(19) United States
(12) Patent Application Publication
(10) Pub. No.: US 2003/0153985 A1
(43) Pub. Date: Aug. 14, 2003

(54) Medical implant

(76) Inventor: Yong Chan Lee, Seoul (KR)

Correspondence Address: MERCHANT & GOULD PC, P.O. BOX 2903, MINNEAPOLIS, MN, 55402-0903, US

(21) Appl. No.: 10/318,465

(22) Filed: Dec. 12, 2002

Related U.S. Application Data
(63) Continuation-in-part of application No. 09/403,234, filed on Dec. 2, 1999, now abandoned.
Application No. 09/403,234 is a 371 of international application No. PCT/KR97/00258, filed on Dec. 5, 1997.

(30) Foreign Application Priority Data
Apr. 26, 1997 (KR) 1997-16481

(51) Int. Cl. A61F 2/28

(52) U.S. Cl. 623/23.59; 433/173; 424/423

(57) ABSTRACT

A medical implant coated with a polymeric material having hydrophilicity and osteoconductivity. When the implant is embedded in depth in a living body, the polymeric material absorbs moisture to swell up, bringing about an effect of dispersing the external stress applied to the implant. This volumetric increase also brings the implant into close contact with the bone of the implantation site, thus remarkably improving the stability in the early stage of the implantation and more strengthening the osseointegration of the implant in the bone as time passes.

### BACKGROUND OF THE INVENTION

1. Field of the Invention

The present invention relates, in general, to a medical implant and, more particularly, to an implant coated with a polymeric material of hydrophilicity and osteoconductivity which enables the implant to secure stability in an early stage of the implantation and to be more strongly integrated in a bone tissue with the lapse of time.

2. Description of the Prior Art

In medicine, an implant is a device intended to be embedded in a tissue for therapeutic purpose or its desired aim. Generally, after an implant is embedded in a predetermined tissue site of a living body, e.g. nasal bone, dental root or other type bones, the integration of the implant is accomplished either through a medium of soft tissue between the implant and the tissue or by filling this site with a bone only without any intermediate. The latter case is called complete osseointegration.

After implantation in a bone tissue, the therapeutic purpose of the implant can be achieved with a higher possibility as the interface between the implant and its surrounding bone is filled with a larger amount of bony material. In conventional techniques, the osseointegration between an implant and a bone was found to be only about 60% and thus, there is a limit in the therapy with implant.

Dental implants have been increasingly used to recover the masticatory function of lost teeth. It is well known that the success of dental implants is heavily dependent on initial stability and long-term osseointegration that leads to optimal stress distribution in the surrounding bones. The role of the periodontal ligament, removed during operation, is to absorb impact force and to distribute it to the alveolar bone. For this reason, the study of the artificial periodontal ligament has become an important issue in this field.

The purpose of the dental implant is to replace lost teeth with artificial material and to achieve structural and functional recovery of the masticatory system. Development of dental implants under the concept of osseointegration began when Bränemark introduced it in 1969. Long term experiments and clinical reports have scientifically proven the efficiency of dental implants and they are currently implemented as the common treatment for the recovery of oral functions.

However, the failure of an implant is largely due to inflammation around the implant and excessive stress around the surrounding bone. The osseointegrated bonding between an implant and its surrounding bone delivers impact to the surrounding bone directly without a change in the amount of load and speed, and persistent pressure by repetitious masticatory movement contributes to this phenomenon.

Furthermore, natural teeth is surrounded by the elastic periodontal ligament, allowing it to have about 100˜200 μm of functional motion in the alveolar bone during mastication. However, implants only have 10 μm of motion because the periodontal ligament was removed during operation.

As a result, a reduction of functional movement causes stress concentration and micro-fractures in alveolar bone. Thus, in a periodontic point of view, it is necessary to reduce the stress concentration and to prevent micro-fracture.

### SUMMARY OF THE INVENTION

It is an object of the present invention to overcome the above problems encountered in prior arts and to provide a medical implant which is greatly improved in osseointegration as well as in stability in the early stage of the implantation.

In accordance with the present invention, there is provided a medical implant which is coated with a polymeric material having hydrophilicity and osteoconductivity both.

### BRIEF DESCRIPTION OF THE DRAWINGS

FIG. 1 shows the surface of the Bränemark implant that is not coated.

FIG. 2 shows the surface of the implant coated with chitosan 10 times.

FIG. 3 shows the surface of the implant coated with chitosan 20 times.

FIG. 4 shows test specimen which implant is implanted into patella bone.

FIG. 5 shows schematic diagram of testing set-up designed for initial stability test.

FIG. 6 shows the model of the whole system with its various components bonded together.

FIG. 7 shows measuring points in mid-section of dental implant.

FIG. 8 represents the displacement on the tip of a dental implant after the rigid body's impact in each case.

FIG. 9 represents the change of stress in the cortical bone(point A).

FIG. 10 represents the change of stress in the cortical bone(point B).

FIG. 11 represents the change of stress in the cortical bone(point C).

FIG. 12 represents the change of stress in the cortical bone(point D).

FIG. 13 shows schema of strain gage installation.

FIG. 14 shows the position of delta rogette gage.

FIG. 15 shows that a chitosan coating is evenly distributed on the surface of titanium implant and a woven bone sticking to chitosan coating is formed.

FIG. 16 illustrates a formation of the wooven bone as shown in FIG. 15.

FIG. 17 illustrates an emergence of chitosan and neovascularization around screws.

FIG. 18 and FIG. 19 show that the surface of titanium implant, the chitosan coating and the formation of the woven bone are combined as if they form one unit.

### DETAILED DESCRIPTION OF THE INVENTION

Chitosan is coated on dental implants to replace the role of intact periodontal ligament in the early phase of osseointegration. It is a natural polymer which can be relatively easily obtained from the nature, shows both hydrophilicity and osteoconductivity. These properties enable chitosan to be used as a medical implant. When chitosan is placed in depth of a body, the polymeric compound absorbs moisture from the body, swelling up. This volumetric increase of the polymeric compound brings about an effect of dispersing the external stress applied to the implant.

In addition, while being volumetrically increased, the polymeric compound placed in depth in a living body allows the implant to come into close contact with the bone, thus exceptionally augmenting the stability of the implant in the early stage of the implantation.

Further, because the polymeric compound embedded together with an implant in a living body displays superior osteoconductivity, the osseointegration of the implant into neighboring bones is further strengthened with the lapse of time.

The new concept of coating an implant's surface with the polymer chitosan is introduced in order to join the implant and the surrounding bone and give the implant the visco-elastic characteristic of the periodontal ligament, the implant. This present invention intends to improve the shock-absorbability of impact and initial stability after the operation of an implant through mechanical testing and 3-dimensional finite element analysis. Studies and comparisons of these processes were aimed at dispersing and mitigating the stress caused by static and impact loading in the surrounding bone tissue.

Mattioli-Belmonte et al (Journal of Bioactive and Compatible Polymers, 1995) discloses coating osteoconductive hydroxyapatite with chitosan and embedding it into bone defects artificially formed in the femurs of rabbits, andhistologically observes the regeneration of bone. The hydroxyapatite is one of calcium phosphate ceramics and usually used as a bone filling material. The hydroxyapatite has an osteoconductive property. However, it is known that it is not osteoinductive. The above researchers assume that the regeneration of bone looks desirable when the surface of osteoconductive material is coated with osteoinductive material. So they coat the osteoconductive hydroxyapatite with chitosan deemed as an osteoinductive material, and embed it into bone defects artificially formed in the femurs of rabbits, which is referred to as an experimental group. On the other hand, the bone defects of a control group are filled with just the hydroxyapatite. As a result of studying the regeneration of bone, the chitosan is a satisfactory interface between bones and the hydroxyapatite, because it promotes an osteoconductive reaction during the filling of bone defects. On the contrary, the control group shows no osteoconductive reaction verifying the osteointegration. Accordingly, the researchers come to the conclusion that the association of chitosan with hydroxyapatite is a step forward in the treatment of bone defects.

As mentioned above, the purpose of Mattioli-Belmonte is to enhance the regeneration of bone by using the chitosan as a coating agent of osteoconductive hydrixtaoatute. However, Mattioli-Belmonte does not disclose that the chitosan is expanded when it contacts the tissue fluid or blood. Also, Mattioli-Belmote relates to a bone filling material using osteoconductivity like other studies using chitosan, and the absorption of stress which is the subject matter of the present invention is not mentioned at all in Mattioli-Belmonte's thesis.

All of other researchers study a bone filling material by using osteoconductive or osteoinductive materials such as calcium phosphate ceramic including hydroxyapatite or bioglass. But, they think the implant is brittle so that it could not absorb any external stress. Also, they have studied the effect of chitosan as osteoconductive implant by using it alone or its combination with other materials. However, the research for absorbing external stress by using the hydrophility of chitosan is not carried out.

On the other hand, the present invention discloses that titanium implant is coated with chitosan and then embedded in jawbone. According to the present invention, the implant obtains remarkably improved stability in the early stage of the implantation because the chitosan can absorb external stress applied to the implant. Therefore, the present invention is different from Mattioli-Belmonte's invention that uses the chitosan just as a bone filling material due to the osteoconductivity of chitosan.

The enhancement of osteoconductivity or osteoinductivity disclosed in the present invention is mentioned just to explain the property of chitosan. That is to say, the object of the present invention is neither to improve the osteointegration nor to use the chitosan as a bone filling material due to the osteoconductivity or osteoinductivity of chitosan.

According to Mattioli-Belmonte, the reason of coating the hydroxyapatite with chitosan is to achieve the osteoconductive process to fill the bone defects by stimulating the bone surrounding the defects because the chitosan is a resorbable compound capable of effecting granulocyte activation.

That is, touse the chitosan as a coating material is to regenerate bone by employing the osteoconductivity of chitosan.

According to the present invention, the reason of coating the surface of titanium implant with chitosan is to use the property of the chitosan as expanding when it contacts tissue fluid or blood in a living body. The expanded material densely fills the space between the implant and neighboring bones, and it can absorb external stress applied to the implant or disperse the stress to the neighboring bones, which is a role of periodontal ligament of natural tooth.

In other words, the object of the present invention is to perform the absorption of stress such as a natural periodontal ligament by coating the implant with the chitosan.

Other researches disclose enhancing the osteoconductivity by filling bone defects with chitosan or the materials combined or coated with chitosan, thereby improving the regeneration of bone.

In contrast, the present invention discloses that titanium implant is coated with chitosan and the coated implant has a temporary role of a periodontal ligament when the implant is placed in a living body. Therefore, the present invention is very different from Mattioli-Belmonte's invention.

In the meantime, it is known that the osteointegration couldn't be achieved in case of coating the surface of implant with a polymer because the polymer can achieve not osteointegration but distant osteogenesis, even though the polymer is biocompatible. Now, some researchers study for enhancing the osteointegration by coating the surface of titanium with osteoinductive protein. However, as described above, these studies are very different from the present invention.

In other words, other researchers think that the osteointegration cannot be achieved if the surface of titanium capable of achieving the osteointegration is coated with a polymer. Thus, none of them coat the surface of titanium with the polymer nor try to restore the temporary role of natural periodontal ligament. The present invention is the first attempt of coating the surface of osteoconductive titanium with a natural polymer and using the chitosan coating as a temporary substitute of a natural periodontal ligament. That is, the present invention is to enhance the osteointegration and to absorb the external stress by coating the implant with chitosan.

Guire (U.S. Pat. No. 4,973,493) discloses providing the surface of biomaterial such as substitute blood vessels, lenses and catheters with a desirable biocompatible agent to improve its biocompatibility. The dental implant which is the subject matter of the present invention is not disclosed anywhere in the Guire's invention. Also, Guire uses such a biocompatible agent as chitosan boned to the surface of biomaterial to prohibit protein or cholesterol deposition to lenses, thereby preventing a bad reaction such as an allergic response.

In contrast, the object of the present invention is to enhance the primary stability of dental implant, and to restore the temporary role of natural periodontal ligament: absorbing external stress applied to the implant an dispersing the stress to the neiboring bones by coating the dental implant with chitosan.

Muzzarelli's thesis disclosed in Biomaterial 14:1 in 1993 is about osteoconductivity of methylpyrrolidinone chitosan.

This article shows how well empty space left from avulsion of the third molar or wisdom teeth is filled up with newly formed bone tissue after being packed with methylpyrrolidinone chitosan made into a form of sponge. There is a feature characterized in that methylpyrrolidinone chitosan has gel-forming ability if it contacts with body fluid in this article.

That is, after avulsion of wisdom teeth, there is an avulsed wound. The avulsed wound has a form of a root so that if methylpyrrolidinone chitosan sponge is hard and brittle, it is difficult to be packed. Thus, in order to be filled in the avulsed wound, in case that the sponge contacts with body fluid, it becomes very soft. By using and combining this hydrophilicity of pyrrolidine, methylpyrrolininone chitosan confers gel-forming ability on the avulsed wound. That is, it is for being packed in tight.

In other words, gel-forming ability, biocompatibility, and osteoconduction in this article are for plasticity of chitosan as bone substitutes.

On the contrary, in the present invention, chitosan is used as a coating material having the shock absorbing property which is one of the roles of periodontal ligament. The shock absorbing is for making a certain device which absorbs an external stress so that a harmful force may not be given to neighboring bone tissues when new bone formation occurs through early process of bone remodeling after embedding an implant.

Rueger et al (U.S. Pat. No. 5,344,654) discloses an osteogenic protein can directly form a new bone as well as enhancing bone in growth when coating an implant with an osteoinductive material which can directly induct the osteogenesis and then embedding the implant in the depth of a bone.

In contrast, the object of the present invention is to protect the bone tissues surrounding a new bone tissue by properly absorbing the external stress through a shock absorbing material.

In case the strong external stress such as chewing food is applied, it has a bad influence on the process of bone remodeling.

Thus, the present invention is to provide a shock absorbing material which can reduce the external stress, thereby making the bone remodeling go smoothly. However, the object of Rueger et al is to form a new bone effectively by coating an implant with an osteoinductive material.

The implant is a material made from titanium, and the bone also has some degree of solidity. When the two materials having different elasticity are in contact with each other, possible external stress brings about complicated physiomechanical reactions. Nevertheless, according to the present invention, the reactions can work favorably in the process of periodontal ligament around bone remodeling by interposing a shock absorbing material between the implant and the bone. The shock absorbing property is an ability of natural dental root. The subject of the present invention is to use chitosan as a shock absorbing material.

A better understanding of the present invention may be obtained in light of following examples which are set forth to illustrate, but are not to be construed to limit, the present invention. In the following examples, prevailing titanium screw type implants in current use were coated with or without chitosan and implanted in living bodies. In either case, the implants were tested for their stability in the early stage of the implantation.

### EXAMPLE 1

### Initial Stability Test

This example was aimed at improving the stability of the dental implant by coating the implant with the polymer layer, chitosan, which was designed to function like a normal periodontal ligament. After coating the Bränemark type implant with chitosan, it was mechanically tested and initial stability between the coated and uncoated implants were compared.

The dip-coating method of chitosan used here involved the following procedures: i) washing out the implant in the acetone solution and then with distilled water. ii) melting chitosan with an acetic solution and reducing it to a 1% solution. iii) dipping the washed implant into the reduced chitosan solution, picking it up at right angles, drying it under vacuum condition for 30 minutes, repeating it 10 to 20 times, and iv) drying the implant under vacuum condition and room temperature for one day.

Fresh patella bones from porcine knees were utilized, since their structure is similar to the alveolar cancellous bone. The soft tissue was removed and the bone was cut into 2 cm×2 cm×2 cm portions with an ISOMET™ (BUEHLER LTD., Lake Bluff, Ill. U.S.A.) for the test.

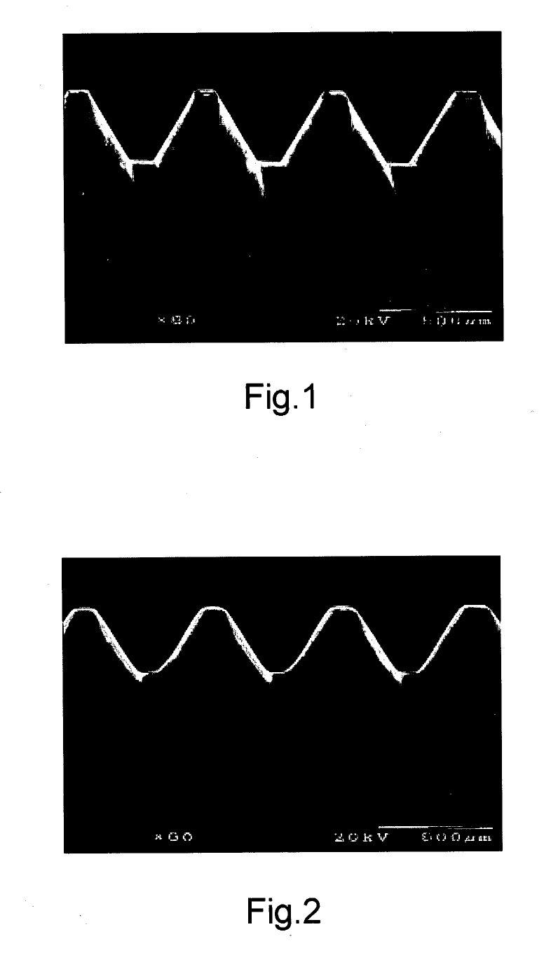

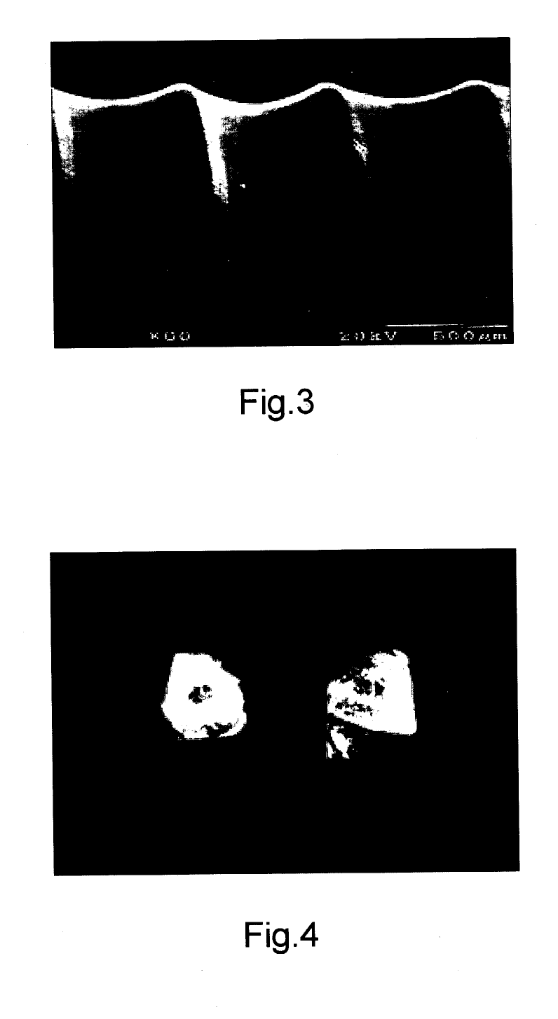

FIG. 1 shows the surface of the Bränemark implant that is not coated. FIG. 2 and FIG. 3 respectively show the surface of the implant coated with chitosan 10 and 20 times. The coated implant with a 30˜50 μm layer of chitosan (FIG. 2) were used in this example. FIG. 4 shows test specimen which implant is implanted into patella bone.

To test initial stability, load was applied to the upper part of an implant in a shear direction (right angles to an implant) with a force of 20N at a constant speed until the surrounding bone was destroyed. Load was applied in a shear direction because early failures of implant operations were mainly due to the destruction of the surrounding bone caused by shear force.

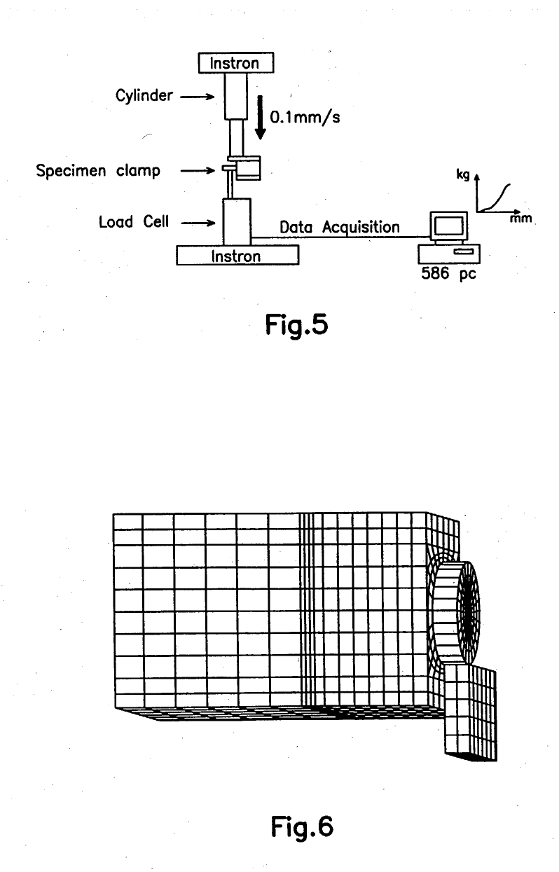

FIG. 5 shows schematic diagram of testing set-up designed for initial stability test. The maximum capacity of the load cell was 50 kg. The speed of the load applied on an implant was 0.1 mm/sec, and the data sampling rate was 20 Hz. The values of the load and displacement were processed by Max V4.0 program.

A load cell located in the lower part of a specimen measured the magnitude of the load. The displacement of an implant was measured with a LVDT(Linear Variable Displacement Transformer) installed in the machine.

A load-displacement curve for each specimen was made. The curve began with a stage of linear behavior until a stage of plastic deformation occurred due to the yield in the surrounding bone of a dental implant. The shear stiffness was derived by regression analysis in the linear region of the load-displacement curve. The limits of the region where the linear slope occurred was arranged so that the coefficient of determination was greater than 0.98. Through this data, the values of shear stiffness, and initial stability, were obtained. The results of shear stiffness in this example were differentiated with the T-test and a p-value was obtained.

The results obtained from the mechanical test of uncoated and chitosan coated implants were shown in Table 1. The uncoated implants had an average shear stiffness of 34.728 kg/mm and the coated implants 47.108 kg/mm.

TABLE 1
Dimension: Kg/mm

| Sample | Uncoated implant | Coated implant |
|---|---|---|
| 1 | 32.519 | 63.087 |
| 2 | 54.218 | 30.895 |
| 3 | 24.035 | 58.201 |
| 4 | 52.697 | 46.919 |
| 5 | 30.404 | 33.772 |
| 6 | 44.800 | 69.154 |
| 7 | 30.020 | 39.016 |
| 8 | 21.980 | 26.009 |
| 9 | 21.500 | 71.826 |
| 10 | 35.960 | 42.172 |
| 11 | 38.560 | 44.695 |
| 12 | 19.252 | 50.578 |
| 13 | 35.549 | 45.951 |
| 14 | 35.403 | 60.900 |
| 15 | 52.939. | 60.567 |
| 16 | 25.321 | 23.632 |
| 17 | 47.925 | 33.460 |
| 18 | 22.022 | Not Available |
| Average | 34.728 | 47.108 |
| S.D. | 11.668 | 14.956 |

| | |
|---|---|
| T-test | p = 1.07E−02 < 0.05 |

In the present invention, the biomechanical experiment was performed by coating a 30˜50 micron thick layer of chitosan on implants. After the operation, the layer would be 50˜100 microns thick, similar to a periodontal ligament's thickness because chitosan expands. Although there was no data about the exact extent of expansion after an operation, histological observation showed chitosan infiltrated into the inner cancellous bone around an implant. Furthermore, chitosan coating adhered well to bone surface without peeling despite the friction caused during operation.

In the table, higher values for the implants mean that the implants are laterally moved in shorter distances when the same force is applied. It is apparent from the table that the displacement of the implants is smaller upon the lateral application of a force of 20N to the implants as they are of larger stiffness.

Coating an implant increased shear stiffness ranging from 11 to 94%, by 35.64% (p<0.05) on the average. It is calculated by the following formula:

(shear stiffness of coated implant−shear stiffness of uncoated implant)*100/shear stiffness of uncoated implant.

Chitosan on implants expanded from its pre-operated dry situation, increasing the stability of implant by filling the gap between the implant and alveolar bone during the implant operation. Furthermore, chitosan substituted the function of the periodontal ligament in the natural tooth, mitigated the load that pressed on the implant, and prevented the yield of surrounding bone.

Therefore, the initial stability of a coated implant was superior to an uncoated implant. The reason why these values were large was because each patella bone used for implants had different anatomical and mechanical characteristics even though they were extracted from the same specimen.

The results of chitosan coating on dental implants demonstrated that it substitutes the function of the removed periodontal ligament. It successfully increased the initial stability of dental implants by 35% that improved the implant's resistance to stress and impacts.

### EXAMPLE 2

### 3-Dimensional Finite Element Analysis of the Impact Loading on Dental Implants

In order to test the shock-absorption effect of the polymer layer on a dental implant, 3-dimensional finite element models were developed for four different cases. Case 1 modeled a control case with natural periodontal ligament a 100 microns thickness. Case 2 modeled an implant with a 50-micron gap in between the alveolar bone. Cases 3 and 4 respectively modeled coated implants with a 50 and 100-micron layer of polymermaterial between the alveolar bone. To shorten analysis time of the finite element model and to simplify modeling, a cylindrical implant was modeled and the form of an alveolar bone, based on the structure of a mandible, was simplified. The finite element model had 4919 nodal points and 4276 eight node brick elements. The material properties of the finite element model in this study are shown in Table 2.

TABLE 2

| | Elastic | | |
|---|---|---|---|
| | modulus | Poisson's | Density |
| Material | (Mpa) | ratio | (g/cm³) |
| Cortical | 13,000 | 0.3 | 2.00 |
| bone | | | |
| Cancellous | 1,300 | 0.3 | 0.97 |
| bone | | | |
| Implant | 110,000 | 0.3 | 4.5 |
| (Titanium) | | | |
| Periodontal | 80 | 0.45 | 1 |
| ligament | | | |
| Chitosan | 150 | 0.45 | 1 |

The material properties of the chitosan layer were obtained from the following procedures. A 1% solution of chitosan was dropped onto a 1% solution of acetic acid on a slide, and made it into a film of regular thickness. It was dried in room temperature for one day, and dried again under vacuum condition. Then the chitosan film was left at a constant temperature and moisture level (25° C., 60%). The tensile strength and the elastic modulus were measured by a standard tensile test using Instron. All materials were assumed to be isotopic and homogeneous. The material properties of the bone structure and the implant were based on information from literature.

Pam-crash™ V96.1 (Engineering System International, Paris, France) program was used for dynamic analysis with elasto-plastic material. Self-contact type 5 was selected for the element and contact type. The boundary between the chitosan layer and bone tissues was assumed to be perfectly bonded. To apply the impact load on the dental implant, a rigid body weighing 0.3 kg was made to collide at a speed of 3 m/s in a lateral direction to the tip of the dental implant. The displacement of the dental implant and the changes of stress in cortical and cancellous bone were calculated. The model of the whole system with its various components bonded together is shown in FIG. 6.

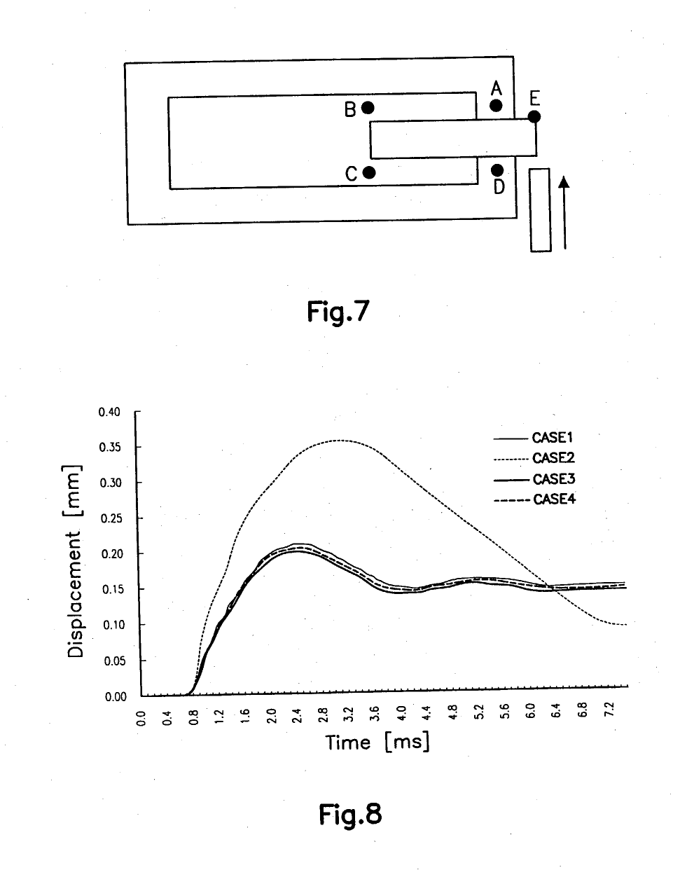

FIG. 7 showed measuring points in mid-section of dental implant. The arrow showed the direction of the rigid body causing impact. Points A, B, C and D were the stress measuring points and point E was the measuring point for the maximum displacement on the tip.

FIG. 8 represents the displacement on the tip of a dental implant after the rigid body's impact in each case. The graph shows that the displacement of case 2 was greater than case 1 because the periodontal ligament absorbed the impact force in case 1. By coating chitosan on the implant in Cases 3 and 4, a displacement curve line similar to Case 1 could be obtained.

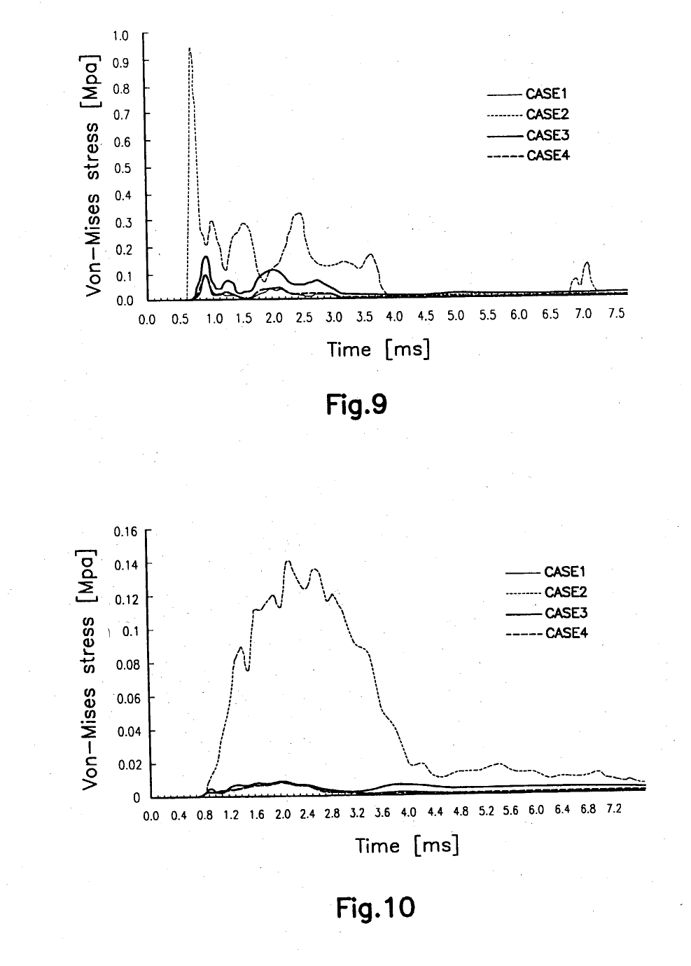

FIGS. 9 and 12 represent the change of stress in the cortical bone (A, D). At point A, the maximum stress generated in Case 2 was 9 times greater than Case 1. The reason was that Case 2 generated the stress by direct transmission from the implant to the surrounding bone without any reduction in impact, whereas in Case 1 the periodontal ligament absorbed the impact. At point D, the stress was less in Case 2 than Case 3 when impact began because only compressive force acted between the implant and the surrounding bone in Case 2 while compressive and tensile forces acted in Case 3. When the implant was coated with chitosan, stress changes at points A and D in Case 4 were more similar to Case 1 rather than in Case 3. This demonstrated that coated implants stabilized more rapidly than uncoated implants.

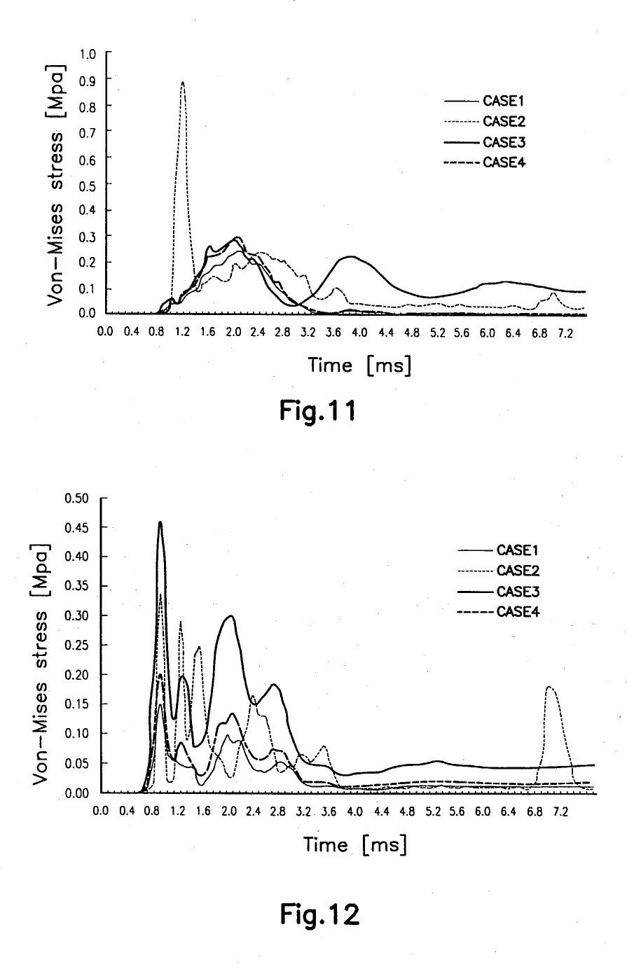

FIGS. 10 and 11 represent the stress change that occurs in the cancellous bone. The stress at points B and C was larger in Case 3 than the other cases. In Case 2, rigid body rotation around the cortical bone resulted in large stress at point C.

For the 3-dimensional finite element analysis, the shape of an implant and its surrounding bone were simplified to save time involved in modeling and calculations. Even though overall stress distribution of the surrounding bone did not precisely correspond to real situations, it does not affect the mutual comparison between the experimental models under the same condition.

In the impact loading experiment, the stress generated in the cortical and the cancellous bone, and the maximum displacement of the implant decreased when the surface of an implant was coated with Chitosan. This showed that the impact stress of the surrounding bone was reduced because the impact energy on an implant was absorbed by the deformation energy of chitosan.

When the results of impact analysis were synthesized, stress on the cortical bone was 10 times greater than stress on the cancellous bone because impact stress was concentrated on the cortical bone and less time was needed to generate maximum stress. In Cases 3 and 4, when maximum stress generated in every observational point was decreased, it resembled the stress change in Case 1. Therefore, methodically coating an implant with a visco-elastic polymer material can reproduce the periodontal ligament's function to absorb the shock of impact reducing the stress convergence generated around the surrounding bone.

The results of FIGS. 9˜12 showed that thicker chitosan coating increased its shock-absorbing function. However, if chitosan thickness is increased indefinitely, the implant loses its function. Implants were intended to be coated with chitosan 20 times in other experimental groups, but a large amount of chitosan peeled off on the surface of an implant during operation. This happened because the coated layer was much thicker than the gap between the implant and the bone tissue.

It is preferable that the coated implant is formed by coating 10˜70 micron thick layer of chitosan on implant. More preferably, the coated implant is formed by coating 30˜50 micron thick layer of chitosan on implant.

FEM modeling and testing showed that stress was distributed more evenly and the stress magnitude was much lower when subjected to impacts, indicating impact adsorption. This increased resistance to stress and impacts can prevent the cause of microfractures in the alveolar bone, benefiting the future of implant technology.

### EXAMPLE 3

### The Effect of a Shock-Absorbable Polymer (Chitosan) Coating of Dental Implant Under Impact Test

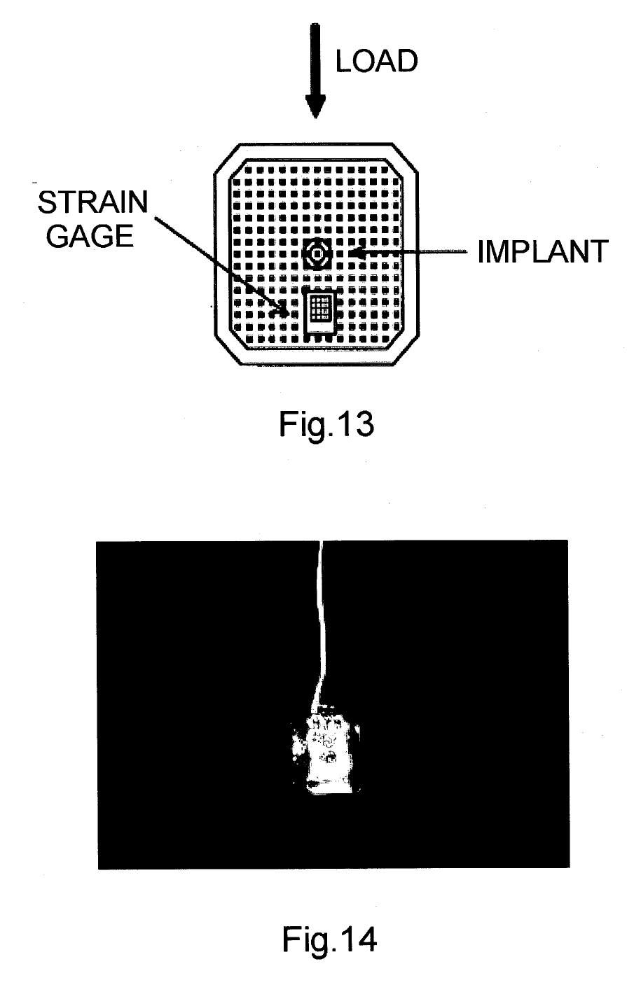

With the object of being provided a temporary artificial periodontal ligament-like membrane, 10 Branemark type implants were coated with commercially available chitosan. These were placed into the fresh patella bones from porcine knees. To test the shock absorbing effect of chitosan coating under impact, three strain gages (Delta-Rosette gage, gage length 1 mm) were located behind an implant as illustrated on FIG. 13. Three gages enable us to measure three linearly independent components of plane strain.

They attached on the cortical bone parallel with the direction of impact force (FIG. 14) and contained three strain gages with the internal angle, i.e. 60 degree respectively. The protocols of impact test were that 1) impact angle is 35 degree obliquely, 2) the gage was located at 6.3 mm from the center of the implant, 3) the length of impact bar was 219 mm and the weight of pendulum was 250 g. We acquired strains(1, 2, and 3) using A/D converter (DATA-Shuttle, strawberry Tree Inc) and its software(Workbench 2.60, sampling rate 50 Hz). Using three strains and the following equation, we could get the principle strain at the time of impact.

$${ϵ}_{p} = \frac{{ϵ}_{1} + {ϵ}_{2} + {ϵ}_{3}}{3} + \frac{1}{\sqrt{3}} \sqrt{{( {ϵ}_{1} - {ϵ}_{2} )}^{2} + {( {ϵ}_{2} - {ϵ}_{3} )}^{2} + {( {ϵ}_{1} + {ϵ}_{3} )}^{2}}$$

On the maximum of principle strain in the direction of impact, there was a significant difference between coated (average 0.064, standard deviation 0.018) and uncoated implants (average 0.095, standard deviation 0.032) (see Table 3). On the delay time of peak strain, the coated implants (average 0.056 sec, standard deviation 0.011 sec) was longer than the uncoated (average 0.024 sec, standard deviation 0.009 sec), statistically (see Table 4).

As the result of our example, chitosan-coated layer could absorb the external impact with small deformation and could delay the time of impact propagation compared with uncoated layer.

TABLE 3

| | Coated | uncoated |
|---|---|---|
| | implant | implant |
| n = 10 | 0.064 | 0.095 |
| | (0.018) | (0.032) |

※ significant at p < 0.05

TABLE 4

| | Coated | Uncoated |
|---|---|---|
| | implant | implant |
| n = 10 | 0.056 | 0.024 |
| | (0.011) | (0.009) |

※ significant at p < 0.05

### EXAMPLE 4

### The Histologic Study on Interface Between Bone and Chitosan Coated Dental Implant—Result of the 21^{st} Day After Transplanting

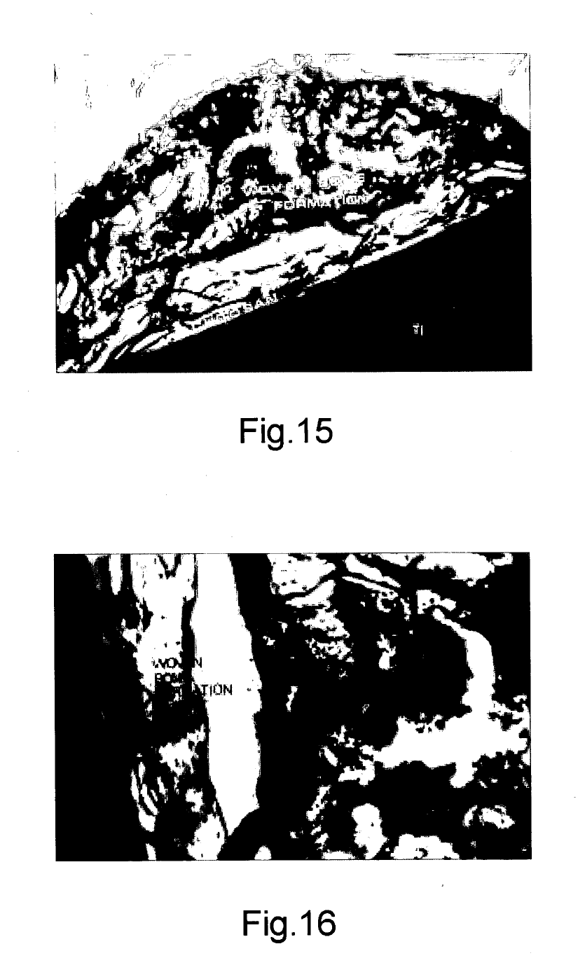

FIG. 15 (×100) shows that a chitosan coating is evenly distributed on the surface of titanium implant and a woven bone sticking to chitosan coating is formed.

FIG. 16 (×200) illustrates a formation of the wooven bone as shown in FIG. 15.

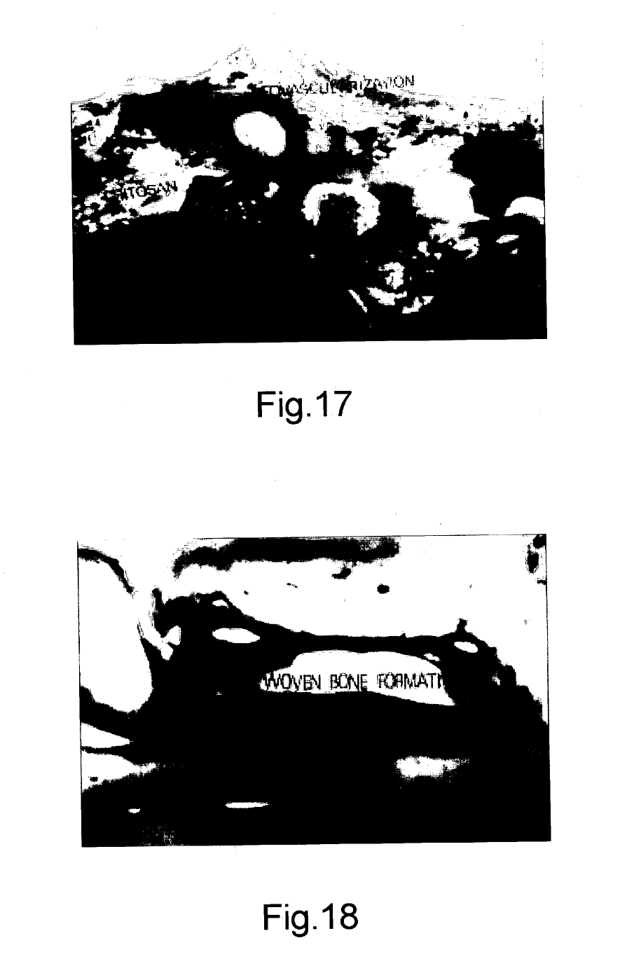

FIG. 17 (×100) illustrates an emergence of chitosan and neovascularization around screws.

FIG. 18 (×200) shows that the surface of titanium implant, the chitosan coating and the formation of the woven bone are combined as if they form one unit.

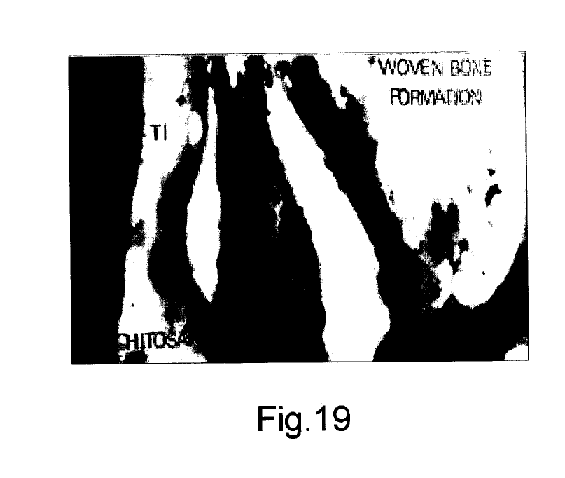

FIG. 19 (×200) shows that the surface of titanium implant, the chitosan coating and the formation of the woven bone are combined as if they form one unit.

Besides chitosan, examples of the naturally occurring compounds which can be as a coat for a medical implant include cellulose polymers, such as cellulose, cellulose acetate, cellulose nitrate and ethyl cellulose, protein polymers, such as gelatin and collagen, guar gum, and cellulose acetate butyrate.

Examples of the synthetic polymers useful as such a coat include poly-L-lactide, poly-D-L-lactide, polyglycolide-lactide, polyacrylonitrile, polyvinylacetate, polyvinylalcohol, poly K-benzyl-L-glutamate, polyphosphazene, polyalkyleneoxalate, polydimethylsiloxane, polyurethane, polyetherurethane amide, polyesterurethane and the mixtures thereof.

As described hereinbefore, when the implant coated with a polymeric material of hydrophilicity and osteoconductivity is embedded in depth in a living body, the polymeric material absorbs moisture to swell up, bringing about an effect of dispersing the external stress applied to the implant. In addition, the implant are brought into close contact with the bone of the graft site by virtue of the volumetric increase and thus, obtains remarkably improved stability in the early stage of the implantation. Furthermore, the polymer of hydrophilicity and osteoconductivity more strengthens the osseointegration of the implant in the bone as time passes.

The present invention has been described in an illustrative manner, and it is to be understood the terminology used is intended to be in the nature of description rather than of limitation.

Many modifications and variations of the present invention are possible in light of the above teachings. Therefore, it is to be understood that within the scope of the appended claims, the invention may be practiced otherwise than as specifically described.

What is claimed is:

1. A method for improving stability of a medical implant in the early stage of implantation by dispersing the external stress applied to the implant comprising the steps of:
selecting a chitosan;
coating a surface of a medical implant with said chitosan; and
embedding the coated implant in a predetermined tissue site of a living body.

2. A method as set forth in claim 1, wherein the coated implant is formed by coating 10˜70 micron thick layer of chitosan on implant.

3. A method as set forth in claim 2, wherein the coated implant is formed by coating 30˜50 micron thick layer of chitosan on implant.

4. A method as set forth in claim 1, wherein the coated implant has significantly initial stability by 11˜94% higher than uncoated implant.
